(19) United States
(12) Patent Application Publication
(10) Pub. No.: US 2020/0385675 A1
(43) Pub. Date: Dec. 10, 2020

(54) METHODS FOR ISOLATING DIFFERENT TYPES OF SINGLE CELLS FROM OVARY

(71) Applicant: SDIVF R&D CENTRE LIMITED, Hong Kong (CN)

(72) Inventors: Zijiang CHEN, Jinan City (CN); Jinlong MA, Jinan City (CN); Yue LV, Jinan City (CN); Han ZHAO, Jinan City (CN); Gang LU, Jinan City (CN)

(73) Assignee: SDIVF R&D CENTRE LIMITED, Hong Kong (CN)

(21) Appl. No.: 16/315,155

(22) PCT Filed: Jul. 3, 2017
(86) PCT No.: PCT/CN2017/091527

§ 371 Date: Mar. 6, 2019

(30) Foreign Application Priority Data
Jul. 4, 2016 (CN) 201610519416.3

(51) Int. Cl. C12N 5/075; C12N 5/0787

(52) U.S. Cl. CPC C12N 5/0609; C12N 2501/734; C12N 5/0642

(57) ABSTRACT

The invention provides a method for separating a mammal early follicle to obtain a single oocyte and a single granulocyte thereof. The method is capable of separating a mammal early follicle to obtain an active single oocyte and a corresponding granulocyte thereof. The invention further provides a kit for obtaining a single oocyte and a single granulocyte thereof from a mammal early follicle.

The present application claims priority to Chinese Patent Application No. 201610519416.3, entitled “Methods for isolating different types of single cells from ovary” filed on Jul. 4, 2016, which is hereby incorporated into the present application by reference in their entirety.

### FIELD OF THE INVENTIONS

The invention relates to the field of cellular biology. In particular, the invention provides a method for treating early follicle of a mammal to obtain single oocyte and single granulosa cells thereof.

### BACKGROUND OF THE INVENTION

Cells are the basic unit of life. Since the introduction of cellular biology, the scientists have been trying to find ways to study cells. In recent years, major breakthroughs in nucleic acid amplification and sequencing technologies have played an important role in the physiological and biochemical research of single cells, making the technology for analyzing single cell a hot research aspect. Therefore, it is particularly important to accurately capture a single cell of specific type for research analysis.

Follicles are the basic structural and functional units of the ovary of a female mammal. Follicles are mainly composed of an oocyte in the center and a surrounding layer or layers of granulosa cells. According to the difference in morphology and function, follicles can be divided into several stages of follicles, including primordial follicles, primary follicles, secondary follicles, preantral follicles, sinusoid follicles and mature follicles. The size of the human follicle can grow from the 30-50 μm in the initial stage to about 20 mm of mature follicles. The mouse primordial follicle is only about 20 μm, and the diameter is about 0.5 mm after maturity. The smallest diameter primordial follicles and primary follicles can be collectively defined as early follicles. At present, the method of separating mammalian follicles in secondary and later stages is very mature and widely used in basic research and clinical treatment. However, there is no uniform and standard method for separating single early follicles, and the method for separating two kinds of cells (i.e., oocytes and their corresponding granulosa) that make up a follicle of primordial follicles and primary follicles is still in the exploratory stage.

For the separation method of follicles, the most commonly used method is enzymatic digestion. Usually, one or two enzymes are used to digest the ovarian tissue. In the digestive mediums, an appropriate amount of DNase can be added to prevent cell adhesion during digestion. During the digestion process, the cells can be separated by shaking or blowing the digestive mediums at intervals, and the digestion is terminated by centrifugal resuspension to obtain follicles. The insufficiency of the existing follicle separation methods is that the separation process often requires a step of resuspending by centrifugation, which has the disadvantage of reducing the recovery efficiency of the cells, and it is easy to cause certain damage to the cells due to improper control of the action of digestive enzymes.

For the separation of single oocytes and corresponding granulosa cells of an early follicle, the existing method is to cut and separate the ovarian tissue after the fixed section by laser capture microdissection (LCM). LCM can accurately cut and obtain individual cells in ovarian tissue. The use of LCM technology to separate individual cells, although very precise, have to fix sections of the tissue prior to the acquisition of active cells. Therefore, live cells cannot be obtained. In addition, the cell structure after sectioning is often incomplete. Therefore, LCM negatively affects subsequent research and analysis on cells.

Therefore, there a need in the art for a method that is more efficient and capable of obtaining live single oocytes and their corresponding granulosa cells in mammals.

### SUMMARY OF THE INVENTION

The present invention for the first time provides a method for efficiently and accurately separating early follicle of mammals and obtaining active single oocyte and single granulosa cell thereof.

The present invention provides a method of obtaining a single oocyte of an early follicle of a mammal and single granulosa cell thereof, comprising the steps of.

(1) Mechanically separating an ovarian tissue of the mammal to small fragments;

(2) digesting the mammalian ovarian tissue fragments in the first digestive medium at about 37° C. for about 20-60 minutes, for example about 30 minutes, wherein said first digestive medium contains collagenase I, collagenase II, collagenase IV or a mixture thereof;

(3) passing the digestive medium mixture obtained in the step (2) through a first pore size cell strainer having a pore diameter of about 40 to 100 μm (for example, about 70 to 80 μm), and then passing the filtrate through a second pore size cell strainer having a pore size of about 8-12 μm (for example, about 8 μm);

(4) rinsing the precipitate in the second pore cell strainer with a culture medium, and then resuspending the precipitate with a culture medium;

(5) aspirating a single early follicle in the resuspended medium;

(6) after washing the obtained single early follicle, digesting it in the second digestive medium for about 3-10 minutes, preferably within 5 minutes, the second digestive medium contains trypsin or Accutase;

(7) transferring the digestive medium mixture obtained in step (6) to a culture medium, then separating and obtaining a single oocyte and single granulosa cells thereof.

The method of the present invention can further comprise the following step (8): using the single oocyte and/or single granulosa cells thereof obtained in step (7) for the following single cell assay, such as cell culture, nucleic acid extraction and analysis, and the like. In one aspect of the present invention, the single oocyte and/or single granulosa cells thereof obtained in step (7) can be directly transferred to culture medium for the following single cell assay.

In the present invention, the early follicles of the mammal refer to primordial follicles and primary follicles. According to the difference in morphology and function, follicles are divided into several periods, including primordial follicles, primary follicles, secondary follicles, preantral follicles, sinusoid follicles and mature follicles. Primitive follicles and primary follicles can be collectively referred to as early follicles. The primordial follicles are often at rest as ovarian reserves, and their structure consists of an oocyte in the center and a layer of flat granule cells wrapped around it. Once activated, the primordial follicles are transformed into primary follicles consisting of an oocyte and a single layer of cubic granule cells. Primordial follicles and primary follicles of different mammalian are relatively similar in volume and structure. The primordial follicles are generally 20-40 μm in diameter and consist of an oocyte of 20 μm in the center and a layer of flat granulosa cells surrounded it. The primary follicles are generally 50-80 μm in diameter and consist of an oocyte of 40 μm in the center and a layer of cubic granule cells surrounding it. Statistics show that the size and structure of primordial follicles and primary follicles in mammals, especially small and medium mammals, are within the above range. Small mammals (generally referred to as mammals having a body length of about 0.5 m or less) include mice, rats, guinea pigs, rabbits, cats, dogs, and the like. Medium-sized mammals (generally referred to as mammals of less than 2 meters in length) include humans, monkeys, caprine species, porcine species, bovine species, and the like.

In step (3), the digested medium in step (2) is passed through a first pore size cell strainer. The pore size of the first pore size cell strainer is selected to be larger than the primary follicle or primordial follicle of the mammal to be treated. The diameter of the strainer allows the target follicle to pass through the first pore cell strainer. The pore size of the first pore size cell strainer is typically from about 40 to 100 μm (e.g., from about 70 to 80 μm).

In step (1), mechanical blunt dissection of the ovarian tissue of the mammal can be performed using conventional medical devices such as forceps, needles, and the like. The ovarian tissue is generally divided into pieces having a size of about 1×10⁶˜9×10⁶ μm³, that is, pieces having a length of about 100 to 300 μm. The mechanical separation is usually carried out in a petri dish containing a dissection solution, or directly in the first digestive medium of step (2).

In one aspect of the invention, the first digestive medium contains a mixture of collagenase I, collagenase II and collagenase IV. In still another aspect of the invention, the first digestive medium contains a mixture of Liberase and collagenase IV. Liberase (such as Liberase™ TL Research Grade, Sigma-Aldrich®, Cat. No. 054010020001) is a widely used digestive enzyme standard reagent in recent years, which contains a mixture of high purity collagenase I and collagenase II. Liberase is free of impurity enzymes such as clostridium protease and trypsin, and is free of >98% of endotoxin and other dead cell components from the raw material. Liberase is commonly used to replace traditional Collagenase I and/or Collagenase II formulations.

In one aspect of the invention, the ratio of Liberase to collagenase IV in the first digestive medium is about 1:4. In still another aspect of the invention, Liberase in the first digestive medium is 0.02 to 0.2 mg/ml, 1o preferably about 0.05 mg/ml, and collagenase IV in the first digestive medium is 0.05 to 0.5 mg/ml, preferably about 0.2 mg/ml.

In the present invention, the first digestive medium may further contain a neutral protease and/or a metalloproteinase such as thermolysin. In still another aspect of the invention, the first digestive medium may further contain DNase.

In one aspect of the invention, the second digestive medium contains trypsin, such as trypsin-EDTA solution (0.25%:0.02%) reagent.

In one aspect of the invention, the second digestive medium contains Accutase (such as StemPro® Accutase® Cell Dissociation Reagent. ThermoFisher, catalog number: A1110501). Accutase is a commonly used digestive enzyme reagent. It has proteolytic and collagenolytic enzymes activities and does not contain mammalian or bacterial derived components. Accutase is often used as a replacement for trypsin. Accutase is a modest digestive reagent and has low damage to cells. In the digestion reaction, single follicles obtained in step (5) are directly added to Accutase solution.

In one aspect of the invention, said second pore size cell strainer is a Transwell. Transwell is a cup-shaped device. There is a permeable polycarbonate membrane on the bottom of the cup, and the rest of the cup is made of the same material as regular cell culture plate. The permeable polycarbonate membrane has micropores with a pore size ranging from 0.4 to 12.0 μm. A transwell having a pore size of 8 μm was used in the preferred embodiment of the present invention method. When the precipitate in the transwell is resuspended in step (4), the transwell is placed right above the liquid below before carrying out step (5).

In the method of the present invention, in step (5), the single follicles in the resuspended solution can be aspirated by using a capillary glass tube or other automated or semi-automatic cell suction device or instrument. After a single follicle is aspirated, the follicle is washed in a clean medium before being transferred to the digestive medium 2 for the step (6).

The present invention also provides a kit for carrying out the method of obtaining a single oocyte of an early follicle of a mammal and single granulosa cell thereof. Said kit contains aforementioned first digestive medium and aforementioned second digestive medium. The compositions of said first digestive medium and second digestive medium are as defined above. For example, in the kit of the present invention, said first digestive medium contains a mixture of collagenase I, collagenase II and collagenase IV. In still another aspect of the invention, the first digestive medium contains a mixture of Liberase and collagenase IV. In one aspect of the invention, the ratio of Liberase to collagenase IV in the first digestive medium is about 1:4. In the present invention, the first digestive medium may further contain a neutral protease and/or a metalloproteinase such as thermolysin. In still another aspect of the invention, the first digestive medium may further contain DNase. In one aspect of the invention, the second digestive medium contains trypsin. In one aspect of the invention, the second digestive medium contains Accutase.

### BRIEF DESCRIPTION OF THE DRAWINGS

FIG. 1 shows separated mice early follicles and their corresponding oocytes and granulosa cells observed under microscope. FIG. 1A shows separated early follicles. FIG. 1B shows separated oocytes and granulosa cells.

FIG. 2 shows oocytes observed under microscope. FIG. 2A shows single oocytes from mouse primordial follicles based on the method in Example 2. FIG. 2B shows single oocytes from mouse primary follicles based on the method in Example 2.

FIG. 3 shows the real-time PCR amplification curve and dissolution curve of the Gapdh gene of a single early oocyte. A: Amplification curve of the single oocyte, B: Dissociation curve of the single oocyte.

FIG. 4 shows gene expression pattern of single oocytes derived from mouse primordial follicles and primary follicles. A: Hierarchical cluster analysis, B: Principal component analysis.

### DETAILED DESCRIPTION OF THE PREFERRED EMBODIMENTS

The nature and benefits of the invention are further described in the following examples, which are intended to illustrate the invention and not to limit the invention.

### Example 1 Preparing Experimental Materials and Animals

Reagent Preparation:

Dissection medium: Leibowitz L15 medium supplemented with 10% fetal bovine serum (FBS) and 100 ug/ml Penicillin-Streptomycin;

Digestion medium 1:

α MEM medium supplemented with 0.05 mg/ml Liberase™ TL Research Grade (Sigma, catalog number: 05401020001, product information: http://www.sigmaaldrich.com/catalog/product/roche/05401020001?lang=zh&region=CN), 0.2 mg/ml collagenase type IV (ThermoFisher, 17104-019) and 5% DNase I Solution (STEMCELL Technologies, 07900).

Digestion medium 2: StemPro® Accutase® Cell Dissociation Reagent (ThermoFisher, catalog number: A1110501, product information: https://www.thermofisher.com/order/catalog/product/A1110501). Accutase is a commonly used digestive enzyme reagent in recent years. It has proteolytic and collagenolytic enzymes activities and does not contain mammalian or bacterial derived components. Accutase is a modest digestive reagent and has low damage to cells. Accutase is often used as a replacement for trypsin.

Culture Medium:

α MEM medium: comprises 10 mIU/mL follicle stimulating hormone (FSH), 3 mg/mL bull serum albumin (BSA), 1 mg/mL Fetuin (Sigma, F3385), 5 mg/mL insulin, 5 mg/mL transferrin, and 5 ng/ml selenium (ITS, Sigma, I3146).

Animals:

One- or two-week female C57 mice

### Example 2 Isolation of Single Oocytes and their Corresponding Granulosa Cells from Mouse Primordial and Primary Follicles

Procedures:

(1) Remove the ovary from mice and suspend in dissection medium and wash 3 times.

Use sterilized needles to mechanically dissociate the ovarian tissue into fragments of approximately 1×10⁶-4×10⁶ μm³ under a dissecting microscope. Transfer to a culture dish containing 2 ml digestion medium 1.

(2) Place the culture dish into a sterile cell incubator and digest at 37° C. for 30 minutes. Mix the digestion medium by pipetting up and down every 10 minutes during digestion. Observing follicle separation by a microscope, when most follicles are in a free state and no large tissue fragments are observed, the next step could be performed.

(3) Filter the medium twice with a 70 μm cell strainer (Falcon® 70 μm Cell Strainer, Corning, 352350). Transfer the filtrate to an 8-μm-pore-size culture insert Transwell (SPLInsert™ Hanging, SPL Life Sciences, 35206) for a further filtration. Discard the filtrate.

(4) Wash the precipitate inside the Transwell 3 times with the culture medium. Then resuspend the precipitate above the membrane with 1 ml of culture medium (Note: during resuspending, the bottom of Transwell needs to be suspended above the liquid level of the culture dish.).

The resuspended culture medium contains follicles and cells of 8 μm and 70 μm in diameter.

The early follicle morphology is as follows:

Primordial follicles (20-40 μm): follicles with an oocyte of about 20 μm in diameter surrounded by a single layer of flattened granulosa cells;

Primary follicles (50-70 μm): follicles with an oocyte of about 40 μm in diameter surrounded by one layer of cubical granulosa cells.

The method of the present invention uses a culture insert with a pore size of 8 μm—Transwell. When the culture insert is placed in a Petri dish, the liquid can easily pass through the membrane, however when the culture insert is suspended right above the medium below, the liquid can stay on the membrane of the culture insert due to surface tension. Using this feature, the method of the present invention is able to re-filter the mixture after the first digestion and first filtration by passing the Transwell and recover the cells retain in the Transwell. The advantages of the method of the present invention include: a. the medium containing digestive enzyme is filtered, digestion can be stopped directly without a step of centrifugation; b. by using a culture insert with a pore size of 8 μm, cells of smaller size such as membrane cells pass through the filter and are discarded; only the early follicles in need are retained. The early follicles can be then resuspended and obtained by aspiration easily without damaging the cells because the liquid staying on the membrane due to surface tension.

(5) Use PicoPipet Micro Pick and Place systems (NEPA GENE) to aspirate the single follicles.

Different diameter S-shaped glass capillaries are selected to pick up early follicles and cells of different sizes: primordial follicles and their corresponding oocytes and granulosa cells need to be picked using a 30 μm S-shaped glass capillary, while primary follicles and their oocytes need to use 50 μm glass capillary. The suction is generally controlled between 0.5 and 1.5V when sucking early follicles and the fine-tuning amplitude should be set to 0.05V. The suction power is increased by 0.5V for cell transferring after the cells reach equilibrium.

(6) After washing the aspirated follicles in the clean medium, transfer them to a container containing digestion medium 2, and then digest them in a cell culture incubator at 37° C. for 5-10 min.

The digestive medium 2 in the method of the present embodiment uses StemPro® Accutase® Cell Dissociation Reagent. Accutase digestive enzyme formulation has a modest and effective digestive enzyme activity that minimizes damage to cells while efficiently digesting and isolating oocytes and granulosa cells in follicles. At the same time, the inventors have found that subjecting the follicles to digestion for a short period of time, for example, within 3 to 5 minutes, is able to minimize damage to the cells, thereby facilitates the direct use of single oocytes and their corresponding granulosa cells after they are separated from follicles in the subsequent experiments on single cells, such as cell culture, nucleic acid extraction and amplification. Longer digestion can easily reduce the activity of oocytes, or even lead to cell degradation.

It was found in the experiments that using trypsin, such as trypsin-EDTA solution (0.25%: 0.02%), can digest follicles and isolate oocytes and granulosa cells. However, after the oocyte and its corresponding granulosa cells are separated into single cells, the step of stopping digestion is required for subsequent single cell experiments, such as termination of trypsin digestion by addition of culture medium for dilution or trypsin inhibitor. Additional steps include washing the cells, or filtering again through a suitable cell strainer.

It has been found that when other digestive enzymes, such as collagenase, are used in the digestive medium 2, it is not possible to effectively digest and separate follicles to oocytes and granulosa cells within a short period of time (for example, within half an hour, especially within 3-10 minutes) required to maintain cell viability.

(7) After the digestion, the mixture in the container holding the digestive medium 2 is pipetted up and down under a microscope (for example, using a capillary tube or a pipette, etc.) until the oocyte and its corresponding granulosa cells are separated into free and single cells.

(8) Single oocytes and/or the granulosa cells thereof are directly transferred to the culture medium for subsequent single cell experiments.

### Example 3 Characterization of Single Oocytes and their Corresponding Granulosa Cells from Mouse Primordial and Primary Follicles

Based on the cell morphology and size of the cells obtained in Example 2, it is confirmed that single oocytes and their corresponding granulosa cells from mouse primordial and primary follicles are obtained.

FIG. 1 shows separated mice early follicles and their corresponding oocytes and granulosa cells observed under microscope. FIG. 1A shows separated early follicles. FIG. 1B shows separated oocytes and granulosa cells.

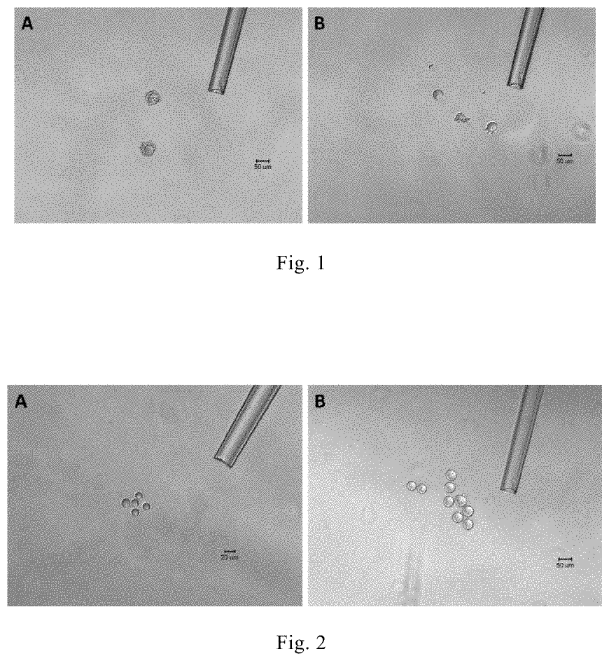

As FIG. 2 shows, single oocytes from mouse primordial and primary follicles are obtained. FIG. 2A shows single oocytes from mouse primordial follicles based on the method in Example 2. FIG. 2B shows single oocytes from mouse primary follicles based on the method in Example 2.

### Example 4 Gene Expression and Hierarchical Cluster Analysis and Principal Component Analysis of Mouse Single Oocytes from Primordial Follicles and Primary Follicles

1. gene expression analysis of mouse single oocytes from primordial follicles and primary follicles obtained in embodiment 2.

Experimental materials: Single Cell Lysis Kit (ThermoFisher, 4458235); SuperScript VILO cDNA Synthesis Kit (ThermoFisher, 1754-050); Platinum Taq DNA Polymerase (ThermoFisher, 10966-034); PowerUp SYBR Green Master Mix (ThermoFisher, A25777).

Experimental Steps:

(1) Transfer the 7 primordial follicle oocytes and 7 primary follicle oocytes isolated in Example 2, respectively, to a PCR tube containing 10 μl Cell Lysis Solution (Single Cell Lysis Kit). Lysis for 5 minutes at room temperature according to the instructions of the kit's manual. The lysate can be stored at −20° C. after lysis.

(2) Add 1 μl of Stop Solution to each PCR tube and stand for 2 minutes at room temperature.

(3) Reverse transcript reaction was performed for each single cells according to the SuperScript VILO cDNA Synthesis Kit instructions. After reverse transcription, 50 μl of denucleated water was added to each tube to dilute the obtained cDNA.

(4) Pre-amplification was carried out using the following specific primers, and the reaction system according to the specification of Platinum Taq DNA Polymerase is as follows:

| | | |
|---|---|---|
| 10*buffer | 2 | μl |
| dNTP | 0.4 | μl |
| MgCl₂ | 0.6 | μl |
| primers mixture | 2 | μl |
| cDNA | 1 | μl |
| Taq polymerase | 0.2 | μl |
| H₂O | 13.8 | μl |
| total | 20 | μl |

PCR Pre-amplification setting is as following:

| | | | | |
|---|---|---|---|---|
| 50° C. | 15 | min | | |
| 70° C. | 2 | min | | |
| 95° C. | 15 | s | | |
| | | | {close oversize brace} | 14 cycles |
| 65° C. | 4 | min | | |

| | |
|---|---|
| 4° C. | ∞ |

(5) the amplicon is used for single cell qPCR. The reaction mixture is as following:

| | |
|---|---|
| Express SYBR GreenER qPCR SuperMix Universal | 5 μl |
| primers (4 μmol/l) | 2 μl |
| DNA | 1 μl |
| DEPC water | 2 μl |
| total | 10 μl |

qPCR is carried out according to instruction of SYBR manual.

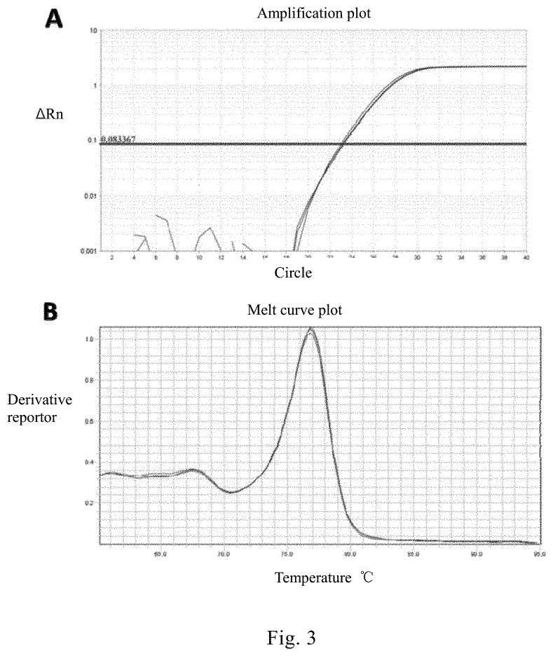

The experimental results can be exemplified in FIG. 3. FIG. 3 shows the real-time PCR amplification curve and dissolution curve of the Gapdh gene of a single early oocyte. The experimental results show that the obtained single oocyte can generate very good qPCR results.

2. Hierarchical cluster analysis and principal component analysis of mouse single oocytes from primordial follicles and primary follicles obtained in Example 2

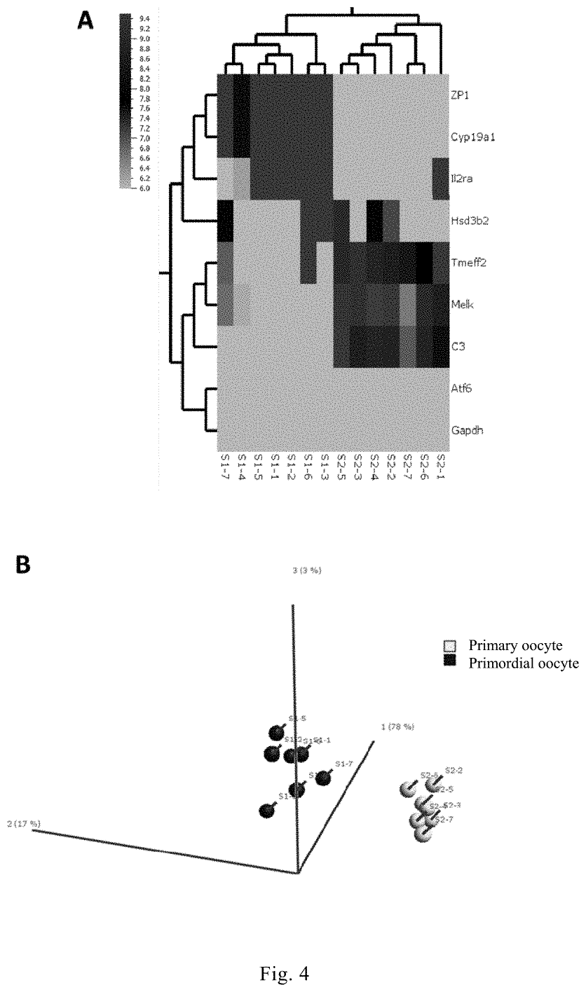

To analysis the single-cell qPCR results of the oocytes from said 7 primordial follicles and 7 primary follicles, CT values obtained by qPCR require direct processing instead of normalization with the reference genes. After setting the effective threshold, deleting the false positive value and processing the missing value, then converting the CT values to the base 2 Log value. Next, import the processed data into Qlucore Omics software for analysis and mapping. The results are shown in FIG. 4. FIG. 4A is a heat map based on gene expression levels of different cells, and it can be seen that the two stages of oocytes each have the highly expressed genes. For example, ZP1 in primordial follicle-derived oocytes is highly expressed compared to primary follicle-derived oocytes. In addition, it can be observed that there is no significant difference in the expression of the housekeeping gene Gapdh in the two kinds of cells. By cluster analysis, it can be observed that the gene expression profiles of primordial follicles and primary follicles are significantly different, which can be used to distinguish between primordial follicles and primary follicles.

FIG. 4B shows the results of different sample principal component analysis. The results are similar to those of the cluster analysis. It can be seen that the principal component analysis results from the primordial follicles and the primary follicle oocytes are significantly different, so it can also be used to distinguish between primordial follicles and primary follicles.

The above results demonstrate that the method of present invention for separating different types of single cells in the ovarian tissue can correctly separate two different early oocytes, namely primordial follicles and primary follicles, and obtain active single cells. Gene expression of oocytes that have lost cell activity is significantly different from gene expression of active cells. The methods of the present invention enable researchers in the field to conduct further studies on single cells. For example, oocytes can be studied in two different developmental stages by selecting genes that are specifically expressed in a certain period of time to understand their different characteristics.

The method of the invention operates at a single follicle, separating the oocyte and corresponding granulosa cells it contains. The method of the present invention reduces the number of manipulations of cells, thereby better protecting the integrity of the cells and enabling a greater number of early follicles to be obtained from the follicles. With the development and maturity of single-cell technology, experiments at the single cell level are receiving increasing attention. Separation of oocytes and their corresponding granulosa cells from viable early follicles in ovarian tissue has been a difficult problem. The method of the present invention successfully solves this problem. It provides a platform basis for subsequent studies of cell activity, gene expression analysis, single cell sequencing and even proteomics from a single early oocyte and corresponding granule cell level.

Unless otherwise indicated, the practice of the present invention will employ common technologies of organic chemistry, polymer chemistry, biotechnology, and the like. It is apparently that in addition to the above description and examples than as specifically described, the present invention can also be achieved in other ways. Other aspects within the scope of the invention and improvement of the present invention will be apparent to the ordinary skilled in the art. According to the teachings of the present invention, many modifications and variations are possible, and therefore it is within the scope of the present invention.

Unless otherwise indicated herein, the temperature unit “degrees” refers to Celsius, namely ° C.

1. A method of obtaining a single oocyte of an early follicle of a mammal and single granulosa cell thereof, comprising the steps of:
(1) blunt separating an ovarian tissue of the mammal;
(2) digesting the mammalian ovarian tissue fragments in the first digestive solution at about 37° C. for about 20-60 minutes, wherein said first digestive solution contains collagenase I, collagenase II, collagenase IV or a mixture thereof;
(3) passing the digestive solution mixture obtained in the step (2) through a first pore size cell strainer having a pore diameter of about 40 to 100 μm and then passing the filtrate through a second pore size cell strainer having a pore size of about 8-12 μm;
(4) rinsing the precipitate in the second pore cell strainer with a culture solution, and then resuspending the precipitate with a culture solution;
(5) aspirating a single early follicle in the resuspended solution;
(6) after washing the obtained single early follicle, digesting it in the second digestive solution for about 3-10 minutes, the second digestive solution contains trypsin or Accutase;
(7) transferring the digestive solution mixture obtained in step (6) to a culture solution, then separating and obtaining a single oocyte and single granulosa cells thereof.

2. The method of claim 1, wherein it further comprises the following step:
(8) using the single oocyte and/or single granulosa cells thereof for single cell assay.

3. The method of claim 1, wherein the early follicle is a primordial follicle or a primary follicle.

4. The method of claim 1, wherein said first digestive solution comprises a mixture of Liberase and collagenase IV.

5. The method of claim 1, wherein said first digestive solution further comprises a neutral protease and/or a metalloprotease.

6. The method of claim 1, wherein said second digestive solution comprises Accutase.

7. The method of claim 1, wherein in step (6), the second digestion is carried out for about 3-5 minutes.

8. The method of claim 1, wherein said second pore size cell strainer is a Transwell.

9. The method of claim 1, wherein the mammal is a small and medium size mammal.

10. A kit for using in a method of obtaining a single oocyte of an early follicle of a mammal and single granulosa cell thereof as defined in claim 1, which comprising a first digestive solution and a second digestive solution, wherein said first digestive solution contains collagenase I, collagenase II, collagenase IV or a mixture thereof, and said second digestive solution contains trypsin or Accutase.

11. The kit of claim 10, wherein the early follicle is a primordial follicle or a primary follicle.

12. The kit of claim 10, wherein said first digestive solution comprises a mixture of Liberase and collagenase IV.

13. The kit of claim 12, wherein said first digestive solution further comprises a neutral protease and/or a metalloprotease.

14. The kit of claim 10, wherein said second digestive solution comprises Accutase.

15. The kit of claim 10, wherein the mammal is a small and medium size mammal.

16. The method of claim 4, wherein said first digestive solution comprises a mixture of Liberase and collagenase IV, wherein the ratio of Liberase to collagenase IV in said first digestive solution is about 1:4.

17. The method of claim 5, wherein said first digestive solution further comprises DNase.

18. The method of claim 9, wherein the mammal is a human being, a monkey, a caprine, a porcine, a bovine, a mouse, a rat, a guinea pig, a rabbit, a cat or a dog.

19. The kit of claim 12, wherein said first digestive solution comprises a mixture of Liberase and collagenase IV, wherein the ratio of Liberase to collagenase IV in said first digestive solution is about 1:4.

20. The kit of claim 13, wherein said first digestive solution further comprises DNase.
